(19) United States
(12) Plant Patent Application Publication
(10) Pub. No.: US 2021/0168985 P1
(43) Pub. Date: Jun. 3, 2021

(54) Dieffenbachia Plant Named 'MAROBA 3'

(50) ;

(71) Applicant: Eduard J.M. van der Knaap, Bleiswijk (NL)

(72) Inventor: Eduard J.M. van der Knaap, Bleiswijk (NL)

(21) Appl. No.: 17/104,575

(22) Filed: Nov. 25, 2020

(30) Foreign Application Priority Data
Nov. 29, 2019 (QZ) 2019/3233

(51) Int. Cl. A01H 6/12

(52) U.S. Cl. PLT/378

(57) ABSTRACT

‘MAROBA 3’ is a new and distinctive variety of Dieffenbachia which is characterized by a compact, upright growth habit with an abundance of dark green, glossy foliage borne on dark green petioles, unique foliage variegation with a moderate amount light greyed-green blotches along the secondary leaf veins with sparse blotches following a similar pattern in the interveinal areas, and the stability of all characteristics from generation to generation.

### CROSS REFERENCE TO RELATED APPLICATIONS

This application claims priority to the Community Plant Variety Rights application number 2019/3233, filed Nov. 29, 2019, which is herein incorporated by reference.

Latin name of the genus and species: The Latin name of the genus and species of the novel variety disclosed herein is Dieffenbachia Schott.

Variety denomination: The inventive variety of Dieffenbachia disclosed herein has been given the variety denomination ‘MAROBA 3’.

### BACKGROUND OF THE INVENTION

Parentage: ‘MAROBA 3’ originated as a naturally-occurring, whole plant mutation of Dieffenbachia ‘MAROBA’ (U.S. Plant Pat. No. 9,220). In August of 2018 the inventor discovered the mutation at a commercial greenhouse in Bleiswijk, the Netherlands, growing amongst a crop of Dieffenbachia ‘MAROBA’. The mutation was noted for its freely-branching, compact growth habit, and unique foliage variegation and was subsequently isolated for further evaluation in order to confirm the distinctness and stability of the characteristics first observed. Upon confirmation of distinctness and stability, the new Dieffenbachia plant was selected for commercialization and given the name ‘MAROBA 3’.

Asexual Reproduction: Asexual reproduction of ‘MAROBA 3’, by way of softwood stem cuttings, was first performed in the November of 2020 at a commercial greenhouse in Bleiswijk, the Netherlands. Five successive generations so produced have shown that the unique features of the instant cultivar are stable and reproduced true to type.

### SUMMARY OF THE INVENTION

The cultivar ‘MAROBA 3’ has not been observed under all possible environmental conditions and the phenotype may vary somewhat with variations in the instant environment such as temperature, day length, and light intensity, without, however, any variance in genotype. The following characteristics have been repeatedly observed and represent the distinguishing characteristics of the new Dieffenbachia cultivar ‘MAROBA 3’. These traits, in combination, distinguish ‘MAROBA 3’ as a new and distinct cultivar.
1. Dieffenbachia ‘MAROBA 3’ exhibits a compact, upright growth habit with an abundance of foliage; and
2. Dieffenbachia ‘MAROBA 3’ exhibits an abundance of dark green, glossy foliage borne on dark green petioles; and
3. Dieffenbachia ‘MAROBA 3’ exhibits a unique foliage variegation pattern consisting of a moderate amount of irregular light greyed-green blotches that generally follow the secondary leaf veins with sparse blotches following a similar pattern in the interveinal areas.

Generally appearing as a darker shade of green, relative to ‘REEVA’; moderately blotched with a mixture of yellow-green and greyed-green along the secondary veins which generally appears nearer to a light a very light greyed-green coloration; interveinal areas are slightly blotched yellow-green.

### BRIEF DESCRIPTION OF THE FIGURES

FIG. 1 shows, as nearly true as it is reasonably possible to make the same in color illustrations of this type, an exemplary ‘MAROBA 3’ plant at approximately 35 weeks old, potted into a 21 cm nursery container, grown in a greenhouse in Bleiswijk, Netherlands.

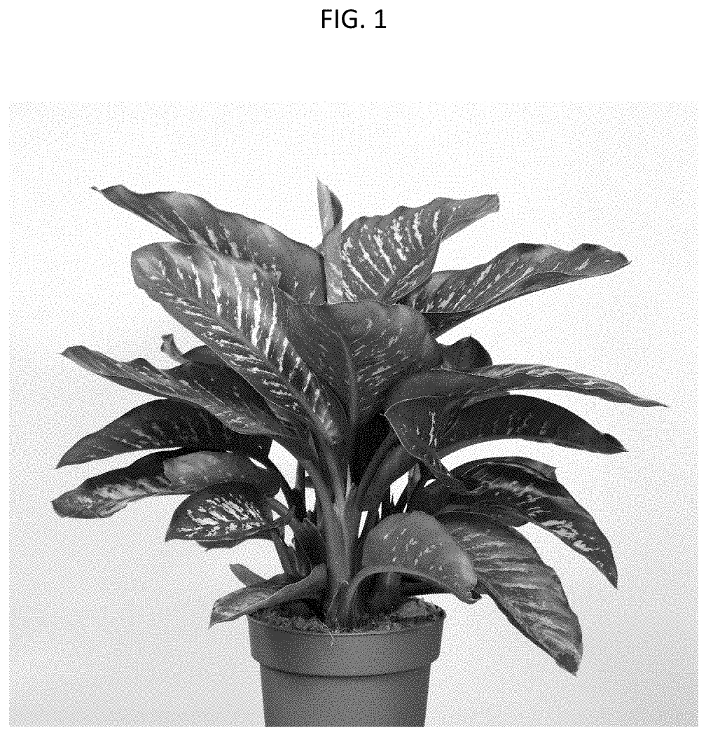

FIG. 2 shows, as nearly true as it is reasonably possible to make the same in color illustrations of this type, the typical foliage of ‘MAROBA 3’.

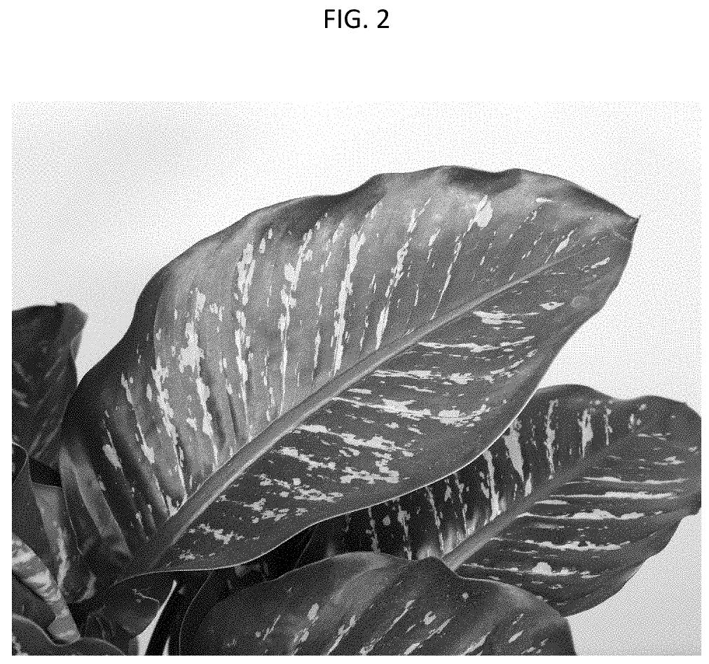

### BOTANICAL DESCRIPTION OF THE PLANT

The following is a detailed botanical description of a new and distinct variety of Dieffenbachia known as ‘MAROBA 3’, based upon observations of plants, approximately 35 weeks old from a rooted cutting, grown in 12 cm nursery container in a climate-controlled greenhouse in Bleiswijk, the Netherlands. Observation data was recorded in November of 2020. Plants were produced using conventional greenhouse production protocols for Dieffenbachia which consisted of regular 2.2 to 3.0 EC fertilizer applications and regular irrigation. No pest or disease control measures were utilized in production. Plants were produced under shade and no photoperiodic treatments or artificial light was given to the plants.

Those skilled in the art will appreciate that certain characteristics will vary with older or, conversely, with younger plants. ‘MAROBA 3’ has not been observed under all possible environmental conditions. Where dimensions, sizes, colors and other characteristics are given, it is to be understood that such measurements are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 2015 edition.

A botanical description of ‘MAROBA 3’ and comparisons with the parent and the most similar variety of common knowledge are provided below.
General plant description:
   Growth habit.—Clump forming, upright perennial.
   Plant form.—Broad ovate to near spherical.
   Growth rate.—Moderately fast to fast growing.
   Plant vigor.—Moderately vigorous.
   Height.—47.7 cm.
   Width.—61.8 cm.
   Propagation.—Softwood stem cuttings.
   Time to initiate roots.—Approximately 21 days to initiate roots at approximately 24 degrees Centigrade.
   Time to produce a finished plant.—Approximately 7 weeks to produce a fully rooted cutting and 30 weeks to produce a finished plant.
   Environmental tolerances.—USDA Zones 10 to 13; at least tolerant of temperatures of up to 40 degrees Celsius. Moderate to high tolerance to rain inundation; low tolerance to wind. Not drought tolerant.
   Pest resistance and susceptibility.—Plants have not been observed to be susceptible or resistant to pathogens and pests common to Dieffenbachia.
Root system:
   General.—Fleshy; sparsely branched, and moderately dense to dense root system.
   Diameter of primary roots.—0.5 cm.
   Color.—Yellow-white, nearest to a mixture of RHS 158B and 158C.
   Texture.—Densely covered with minute root hairs.
Stems:
   Branching habit.—Main shoots arise directly from the soil and only occasionally branch.
   Number of main shoots per plant.—5.
   Number of lateral branches per plant.—1.
   Appearance; cross-section.—Rounded.
   Length of main shoots.—14.0 cm.
   Diameter of main shoots.—4.3 cm.
   Internode length on main shoots.—2.4 cm.
   Aspect.—Erect; near vertical.
   Strength.—Strong.
   Texture and luster.—Glabrous, smooth and moderately glossy.
   Color, juvenile.—Yellow-green, nearest to RHS 145C.
   Color, mature.—Yellow-green, nearest to RHS 145A.
   Color at internodes.—Yellow-green, nearest to RHS 145A.
Foliage:
   Arrangement.—Alternate.
   Division.—Simple.
   Quantity of leaves per lateral branch.—Average of 10.
   Leaf shape.—Ovate.
   Leaf apex.—Apiculate with an abruptly acute tip.
   Leaf base.—Truncate.
   Aspect.—Slightly to moderately carinate, slightly reflexed.
   Attitude.—Upward and outward.
   Leaf length.—Average 29.8 cm in length.
   Leaf width.—Average 16.9 cm in width.
   Leaf margin.—Entire; moderately undulate.
   Texture, adaxial surface.—Glabrous, glabrous and moderately glossy.
   Texture, abaxial surface.—Glabrous, glabrous and slightly glossy.
   Juvenile color, adaxial surface.—Nearest to in between green and yellow-green, RHS 137B and 146A; areas surrounding the secondary veins are heavily blotched and suffused yellow-green, nearest to a combination of RHS 145C, 145D, and 150D; interveinal areas are slightly blotched yellow-green, nearest to RHS 147D.
   Juvenile color, abaxial surface.—Nearest to yellow-green, RHS 147B; areas surrounding the secondary veins are heavily blotched and suffused with a mixture of yellow-green and greyed-green, nearest to RHS 144B, 150D, and 192A; interveinal areas are slightly blotched yellow-green, nearest to RHS 144B.
   Mature color, adaxial surface.—Nearest to in between green and yellow-green, RHS NN137B and 147A; areas surrounding the secondary veins are moderately blotched with a mixture of yellow-green and greyed-green, nearest to RHS 148C, 148D, and 194B; interveinal areas are very slightly blotched with a mixture of yellow-green and greyed-green, nearest to RHS 148C, 194A, and 194B.
   Mature color, abaxial surface.—Green, nearest to RHS 137B; areas surrounding the secondary veins are moderately blotched with a mixture of yellow-green and greyed-green, nearest to RHS 148D and 193A; interveinal areas are very slightly blotched with a mixture of yellow-green and greyed-green, nearest to RHS 148D and 193A.
   Venation.—Vein pattern — Pinnate. Vein color, adaxial surface — Green, nearest to RHS 137A. Vein color, abaxial surface — Nearest to a mixture of green and yellow-green, RHS 143A and 144B.
   Petiole.—Length — Approximately 2.3 cm, excluding the petiole wing. Diameter — Petioles are subterete with a somewhat flattened adaxial surface; average width is 1.05 cm; average height is 1.2 cm. Texture and luster, adaxial surface — Glabrous, smooth, and glossy. Texture and luster, abaxial surface — Glabrous, smooth and slightly glossy. Color, adaxial surface — Green, nearest to RHS NN137A. Color, abaxial surface — Nearest to a mixture of green and yellow-green, RHS NN137A and 144A. Strength — Strong. Geniculum — None present. Petiole wings — Length — Approximately 13.5 cm. Width — Approximately 1.4 cm. Color, outer surface — Yellow-green, nearest to RHS 146D. Color, abaxial surface — Yellow-green, nearest to RHS 146B and fading lighter distally, nearest to RHS 145A.
Inflorescence: No flowers observed to date.
Flower bud: No flowers observed to date.
Flower: No flowers observed to date.
Reproductive organs: No flowers observed to date.
Seed and fruit: No flowers observed to date.

### COMPARISONS WITH THE PARENTS

Plants of the new cultivar ‘MAROBA 3’ differ from its parent, Dieffenbachia ‘MAROBA’ (U.S. Plant Pat. No. 9,220), by the characteristics described in Table 1.

TABLE 1

| Characteristic | ‘MAROBA 3’ | ‘MAROBA’ |
|---|---|---|
| Foliage size | Smaller than ‘MAROBA’. | Larger than MAROBA 3'. |
| General coloration of the | A mixture of green and | Yellow-green, generally |
| juvenile foliage; adaxial | yellow-green, generally | appearing as a lighter |
| surface. | appearing as a darker shade | shade of green; |
| | of green, relative to | moderately blotched with a |
| | ‘MAROBA’; heavily blotched | mixture of green and |
| | yellow-green which generally | yellow-green which |
| | appears nearer to a light a | generally appears nearer |
| | very light green coloration; | to a yellow or green-yellow |
| | interveinal areas are slightly | coloration. Irregularly, |
| | blotched yellow-green. | broadly margined green. |
| General coloration of the | A mixture of green and | A mixture of green and |
| mature foliage; adaxial | yellow-green, generally | yellow-green, generally |
| surface. | appearing as a darker shade | appearing as a lighter |
| | of green, relative to | shade of green; more |
| | ‘MAROBA’; moderately | heavily blotched with a |
| | blotched with a mixture of | mixture of yellow-green |
| | yellow-green and greyed- | and greyed-green which |
| | green along the secondary | generally appears nearer |
| | veins which generally | to a yellow or green-yellow |
| | appears nearer to a very | coloration. Irregularly, |
| | light greyed-green coloration; | broadly margined with a |
| | interveinal areas are slightly | mixture of yellow-green |
| | blotched yellow-green. | and green. |
| General coloration | Darker shade of green, | Lighter shade of green, |
| of the main veins. | relative to ‘MAROBA’. | relative to ‘MAROBA 3’. |

### COMPARISONS WITH THE MOST SIMILAR VARIETY OF COMMON KNOWLEDGE

Plants of the new cultivar ‘MAROBA 3’ may be distinguished from the commercial variety Dieffenbachia ‘REEVA’ (U.S. Plant Pat. No. 29,666) by the combination of characteristics described in Table 2.

TABLE 2

| Characteristic | ‘MAROBA 3’ | ‘REEVA’ |
|---|---|---|
| Foliage size | Smaller than ‘REEVA’. | Larger than ‘MAROBA 3’. |
| General coloration | Darker shade of green, | Lighter shade of green, |
| of the petiole. | relative to ‘REEVA’. | relative to ‘MAROBA 3’. |
| General coloration of the | Generally appearing as a | Generally appearing as a |
| juvenile foliage; adaxial | darker shade of green, | lighter shade of green; |
| surface. | relative to‘REEVA’; heavily | heavily, centrally blotched |
| | blotched yellow-green | with a mixture of green and |
| | along the secondary veins, | yellow-green which |
| | which generally appears | generally appears as a light |
| | nearer to a very light green | yellow-green. |
| | coloration; interveinal | |
| | areas are slightly blotched | |
| | yellow-green. | |
| General coloration of the | Generally appearing as a | Generally appearing as a |
| mature foliage; adaxial | darker shade of green, | lighter shade of green; |
| surface. | relative to ‘REEVA’; | heavily, centrally blotched |
| | moderately blotched with a | with a mixture of green and |
| | mixture of yellow-green | yellow-green which |
| | and greyed-green along | generally appears as a light |
| | the secondary veins which | yellow-green. |
| | generally appears nearer | |
| | to a very light greyed-green | |
| | coloration; interveinal | |
| | areas are slightly blotched | |
| | yellow-green. | |
| General coloration | Darker shade of green, | Lighter shade of green, |
| of the main veins. | relative to ‘MAROBA’. | relative to ‘MAROBA 3’. |

That which is claimed is:

1. A new and distinct variety of Dieffenbachia plant named ‘MAROBA 3’, substantially as described and illustrated herein.
